(19) United States
(12) Patent Application Publication
(10) Pub. No.: US 2020/0138966 A1
(43) Pub. Date: May 7, 2020

(54) BIFUNCTIONAL SMALL PEPTIDE FOR AUTOIMMUNE DIABETES

(71) Applicants: Board of Supervisors of Louisiana State University and Agricultural and Mechanical College, Baton Rouge, LA (US); INNOLYZER LABS, L.L.C., Shreweport, LA (US)

(72) Inventors: Christopher KEVIL, Shreveport, LA (US); John CHIDLOW, Shreveport, LA (US); Kevin PAVLICK, Shreveport, LA (US)

(21) Appl. No.: 16/480,708

(22) PCT Filed: Jan. 29, 2018
(86) PCT No.: PCT/US2018/015692

§ 371 Date: Jul. 25, 2019

Related U.S. Application Data
(60) Provisional application No. 62/451,093, filed on Jan. 27, 2017.

(51) Int. Cl. A61K 47/62; A61K 38/28; A61K 35/39

(52) U.S. Cl. CPC A61K 47/62; A61K 35/39; A61K 38/28

(57) ABSTRACT

The present invention is related to methods and pharmaceutical compositions comprising a Slit-2 and SDF-1α bifuncitional peptide conjugate having a Slit-2 ligand binding domain and an SDF-1α ligand binding domain.

### CROSS REFERENCE TO RELATED APPLICATIONS/PRIORITY

The present invention claims priority to U.S. Provisional Patent Application No. 62/451,093 filed Jan. 27, 2017, which is incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### BACKGROUND

There is a persistent and devastating lack anti-diabetic treatments for Type 1 diabetes (T1D). T1D is a ravaging autoimmune disease, characterized by destruction of the insulin producing pancreatic β cells leading to insulin deficiency and hyperglycemia. Most importantly, the autoimmune T cell infiltration is a silent killer of pancreatic β cells and by the time of diagnosis a majority of β cells are already lost. Furthermore, persistent glycemic control and insulin replacement therapy is crucial for the T1D patients. Often chronic hyperglycemia in T1D patients causes severe complications such as cardiovascular diseases, renal disease, diabetic retinopathy and diabetic neuropathy sooner or later in their life. The most vulnerable population of T1D is children aged 14 or less. In 2015, more than half a million children were reported to be living with T1D, with the second highest percentage living in the US. Furthermore, incidence of T1D is increasing globally by 3% every year, with reports of 86,000 children developing T1D each year. Although, several disease-modifying strategies have been pursued to address the issues of immunological defects associated with T1D, there has been limited success to find effective therapeutics. A recent report by global data analysts estimated that the global T1D drug market is projected to double from $6.6 billion to $13.6 billion by 2023, yet even with such market pressure, a sufficient therapy has yet to be developed. The current treatment regimen for T1D includes aggressive management of the blood sugars and insulin replacement therapy. Further, in some cases of T1D treatments including beta cells or pancreatic transplantation have been reported. However, the number of donors and tissues limits pancreatic transplantation. Furthermore, current therapies lack treatments that either cure the disease or at least delay its progression. Thus, there is an urgent need for the development of therapies aimed at selectively preventing the beta cell destruction to prevent the T1D progression.

### SUMMARY

Wherefore, it is an object of the present invention to overcome the above mentioned shortcomings and drawbacks associated with the current technology. The present invention is directed to methods and compositions for treating T1D that satisfy the above shortcomings and drawbacks.

The present invention relates to pharmaceutical compositions of a therapeutic (e.g., Slit-2 and SDF-1α bifuncitional peptide conjugates, “SaA-biF-PCs”), or a pharmaceutically acceptable salt, solvate, or prodrug thereof, and use of these compositions for the treatment of T1D.

In some embodiments, the therapeutic, or a pharmaceutically acceptable salt, solvate, or prodrug thereof, is administered as a pharmaceutical composition that further includes a pharmaceutically acceptable excipient.

In some embodiments, administration of the pharmaceutical composition to a human results in a peak plasma concentration of the therapeutic between 0.05 μM-10 μM (e.g., between 0.05 μM-5 μM).

In some embodiments, the peak plasma concentration of the therapeutic is maintained for up to 14 hours. In other embodiments, the peak plasma concentration of the therapeutic is maintained for up to 1 hour.

In some embodiments, the condition is T1D.

In certain embodiments, the T1D is mild to moderate T1D.

In further embodiments, the T1D is moderate to severe T1D.

In other embodiments, the therapeutic is administered at a dose that is between 0.05 mg-5 mg/kg weight of the human.

In certain embodiments, the pharmaceutical composition is formulated for oral administration.

In other embodiments, the pharmaceutical composition is formulated for extended release.

In still other embodiments, the pharmaceutical composition is formulated for immediate release.

In some embodiments, the pharmaceutical composition is administered concurrently with one or more additional therapeutic agents for the treatment or prevention of the T1D.

In some embodiments, the therapeutic, or a pharmaceutically acceptable salt, solvate, or prodrug thereof, is administered as a pharmaceutical composition that further includes a pharmaceutically acceptable excipient.

In some embodiments, administration of the pharmaceutical composition to a human results in a peak plasma concentration of the therapeutic between 0.05 μM-10 μM (e.g., between 0.05 μM-5 μM).

In some embodiments, the peak plasma concentration of the therapeutic is maintained for up to 14 hours. In other embodiments, the peak plasma concentration of the therapeutic is maintained for up to 1 hour.

In other embodiments, the therapeutic is administered at a dose that is between 0.05 mg-5 mg/kg weight of the human.

In certain embodiments, the pharmaceutical composition is formulated for oral administration.

In other embodiments, the pharmaceutical composition is formulated for extended release.

In still other embodiments, the pharmaceutical composition is formulated for immediate release.

The present invention is related to methods and pharmaceutical compositions comprising a Slit-2 and SDF-1α bifuncitional peptide conjugate having a Slit-2 ligand binding domain and an SDF-1α ligand binding domain. Alternative embodiments include comprising a linker that spans about 40 A°. Alternative embodiments include a linker is one of (Acp-G-Acp-G-Acp)₂ and (PEG3-PEG3)₂, where “Acp” is Amino caproate, “G” is Glycine, and “PEG” is Polyethylene Glycol. Alternative embodiments include the SDF-1α ligand binding domain including AYWKENKEQ. Alternative embodiments include the Slit-2 ligand binding domain containing an LLR domain. Alternative embodiments include the Slit-2 ligand binding domain containing one of TITEIRLEQN, LRRIDLSNN, LNSLVLYGN, LQLLLLNAN, and LNLLSLYDN. Alternative embodiments include a peptide sequence of the Slit-2 and SDF1α bifuncitional peptide conjugate includes one of Ac-TITEIRLEQN-(Acp-G-Acp-G-Acp)₂-AYWKENKEQ-NH₂, Ac-LRRIDLSNN-(Acp-G-Acp-G-Acp)₂-AYWKENKEQ-NH₂, Ac-LNSLVLYGN-(Acp-G-Acp-G-Acp)₂-AYWKENKEQ-NH₂, Ac-LQLLLLNAN-(Acp-G-Acp-G-Acp)₂-AYWKENKEQ-NH₂, Ac-LNLLSLYDN-(Acp-G-Acp-G-Acp)₂-AYWKENKEQ-NH₂, and AC-LQLLLLNAN-(PEG₃-PEG₃)₂-AYWKENKEQ-NH₂, where “Acp” is Amino caproate, “G” is Glycine, and “PEG” is Polyethylene Glycol. Alternative embodiments include the peptide conjugate is one acetylated at an N- terminus, amidated at a C- terminus, and both acetylated at the N- terminus and amidated at the C- terminus. Alternative embodiments include the peptide conjugate contains a peptide mimetic of SLIT2. Alternative embodiments include the peptide conjugate contains a peptide mimetic of SDF-1α.

The present invention further relates to compositions and methods of treating Type 1 diabetes comprising administering to a mammalian patient a thereapeutic amount of pharmaceutical composition including a Slit-2 and SDF-1α bifuncitional peptide conjugate. Alternative embodiments include the step of administering insulin to the patient. Alternative embodiments include transplanting one of beta cells or a pancreas to the patient.

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore the claimed invention should not necessarily be construed as limited to addressing any of the particular problems or deficiencies discussed herein.

### BRIEF DESCRIPTION OF THE DRAWINGS

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIG. 1 is a schematic representation of the bifunctional peptide conjugate principle according to one embodiment of the present invention, showing a conjugate peptide containing the SDF-1□ peptide agonist region that binds to CXCR4 and a Slit-2 peptide from the LRR domain that binds Robo-1. In this way, the bifunctional peptide is able to simultaneously bind both receptors to simulate diabetogenic T cell chemorepulsion.

FIG. 2 is a bar graph showing that SDF-1 selectively stimulates diabetogenic T cell chemorepulsion of NOD/LtJ mice and patients with T1D. This graph shows SDF-1 dependent percent change in adhesion of CD3 T cells from C57BL, NON/LtJ, or NOD/LtJ mice to TNF-a activated islet microvascular endothelial cells. *p<0.05 vs control, \hat{ }p<0.05 increase vs TNF-a treatment, #p<0.05 decrease vs TNF-a treatment.

FIG. 3 is a line graph showing non-diabetic human CD3 T cell shear mediated detachment from fresh frozen human pancreas tissue with or without SDF-1 pretreatment.

FIG. 4 is a line graph showing T1D human CD3 T cell detachment from fresh frozen human pancreas tissue with or without SDF-1 pretreatment. 3 non-diabetic and 3 T1D subjects <1 yr diagnosis were used for T cell isolations. *p<0.01 w/o vs with SDF-1 treatment.

FIG. 5 is a schematic representation showing SDF-1 stimulates shear mediated detachment of NOD CD3 T cells in a Robo-1 dependent manner.

FIG. 6 is a bar graph showing that SDF-1 stimulates NOD T cell shear detachment at 1 dyne/cm². *p<0.05 between comparisons.

FIG. 7 is a bar graph showing that SDF-1 stimulates NOD T cell shear detachment at 2.5 dynes/cm². Anti-Robo-1reverses SDF-1 mediated shear detachment. *p<0.05 between comparisons.

FIG. 8 is a bar graph showing CXCR4 protein expression in NOD/LtJ mice over time.

FIG. 9 is a bar graph showing Robo-1 protein expression in NOD/LtJ mice over time.

FIG. 10 is a graph and photos of CXCR4 protein expression in human in patients with T1D.

FIG. 11 is a graph and photos of Robo-1 protein expression in human in patients with T1D.;

FIG. 12 is a schematic representation showing Slit-2/SDF-1 dependent NOD CD3 T cell shear mediated detachment.

FIG. 13 is a bar graph showing NOD CD3 T cell shear detachment at 1 dyne/cm² to adhesion molecules with SDF-1 (250 ng/ml) ±Slit-2 (20 μg/ml). *p<0.05 between indicated comparisons.

FIG. 14 is a bar graph showing NOD CD3 T cell shear detachment at 2.5 dyne/cm² to adhesion molecules with SDF-1 (250 ng/ml) ±Slit-2 (20 μg/ml). *p<0.05 between indicated comparisons.

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm. The embodiments set forth the below represent the necessary information to enable those skilled in the art to practice the invention and illustrate the best mode of practicing the invention. In addition, the invention does not require that all the advantageous features and all the advantages need to be incorporated into every embodiment of the invention.

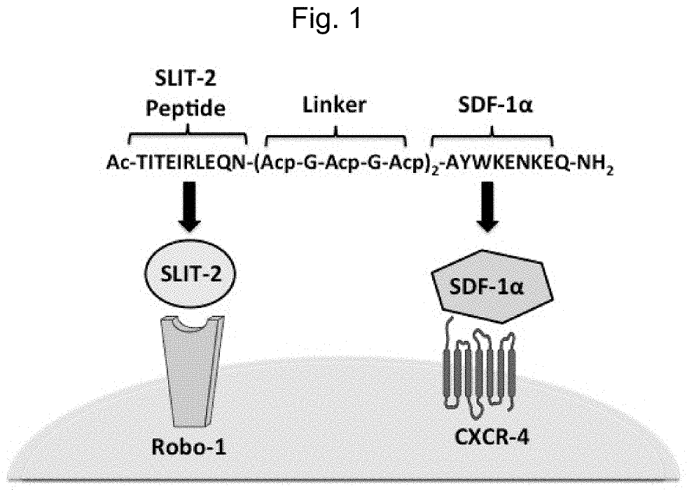

Turning now to FIG. 1, a brief description concerning the various components of the present invention will now be briefly discussed. The inventors disclose herein Slit-2 and SDF1α Bi Functional Peptide Conjugates (SaS-biF-PCs), novel bi-functional peptide conjugates to selectively modulate immune response in T1 D by disrupting autoimmune T cell recruitment to the pancreas. The inventors recently revealed a novel mechanism of differential autoimmune leukocyte recruitment involving selective chemorepulsion of diabetogenic T cells through the actions of SDF-1α/CXCR4 and Slit-2/Robo-1 (U.S. Pat. No. 9,234,886, incorporated herein by reference). However, such approach requires significant amounts of peptide agonists in order to sufficiently bind individual cell surface receptors for chemorepulsion.

The present invention discloses using bifunctional peptide conjugates consisting of discrete ligand binding regions of Slit-2 and SDF1α linked together to selectively modulate T1 D T-cell chemorepulsion. As shown in FIG. 1, the novel bi-functional protein conjugates consist of two small binding peptides, Slit-2 and SDF1α, connected to each other via non-substrate linker. Further, the simultaneous engagement of Slit-2 and SDF1α Bi Functional Peptide Conjugates with their respective receptors Robo-1 and CXCR-4 on diabetogenic T cells to selectively prevent T cell adhesion to pancreatic endothelium is expected to effectively diminish autoimmune diabetes. The inventors believe that SaS-biF-PCs are potent and efficient anti-diabetic therapeutic agents that could increase response rates and reduce treatment time. Also, SaS-biF-PCs can be used in conjugation with insulin replacement therapies as they can help rescue residual beta cells mass left at the time of diagnosis due to the effect of SDF-1α stimulation of beta cell proliferation, and delay progression of disease. Moreover, protein therapeutics have a major advantage over the small molecule therapies; proving to be more highly specific leading to reduced adverse effects. In addition, SaS-biF-PCs can be used in conjunction with either beta cells or pancreatic transplantation therapies to prevent autoimmune T cell infiltration into newly implanted tissues.

The T1D treatments currently available do not focus on prevention or treating root cause of disease. Although, human insulins and analogues dominate the market to date, the largest unmet need for patients is the lack of therapies that would disrupt autoimmune cells and interfere with T1D pathogenesis. The inventors focus is on the unmet need of the patients and provides a novel product that can inhibit T1D pathogenesis.

Development of autoimmune diabetes is now widely appreciated to involve multiple aspects of immunological dysfunction including but not limited to aberrant T cell activation to auto-antigen, altered immune tolerance responses, and defective regulatory immune control. Multiple intervention strategies have been devised to address these issues, however there is not a single FDA approved treatment available to prevent insulitis and β cell destruction. The most important aspect of T1D pathogenesis is autoimmune cell trafficking and adhesion to the pancreatic tissues. There is an unmet need of therapies that selectively regulate of autoimmune cell recruitment in pancreas.

The Slit-2 and SDF1 α Bi-Functional Peptide (SaS-biF-PCs) are novel peptide conjugates based on the inventors' discovery that SDF-1 α stimulated diabetogenic T cell chemorepulsion is in response CXCR4 and Robo-1 co-stimulation. Slit-2 and SDF1 α Bi-Functional Peptide Conjugates molecules are the first types of molecules designed to bind simultaneously to Robo-1 and CXCR-4 receptors respectively on the surface of T cells to prevent T cell adhesion and recruitment in T1D. The disclosed bi-functional conjugate molecules have been designed to selectively modulate autoimmune T cell immune trafficking with minimal potential impact on general immune responses; which significantly differs from previous and current treatment approaches that can leave a patient's immune system compromised. SaS-biF-PCs designed in this study are believed to be able to control T1D in an efficient and selective way.

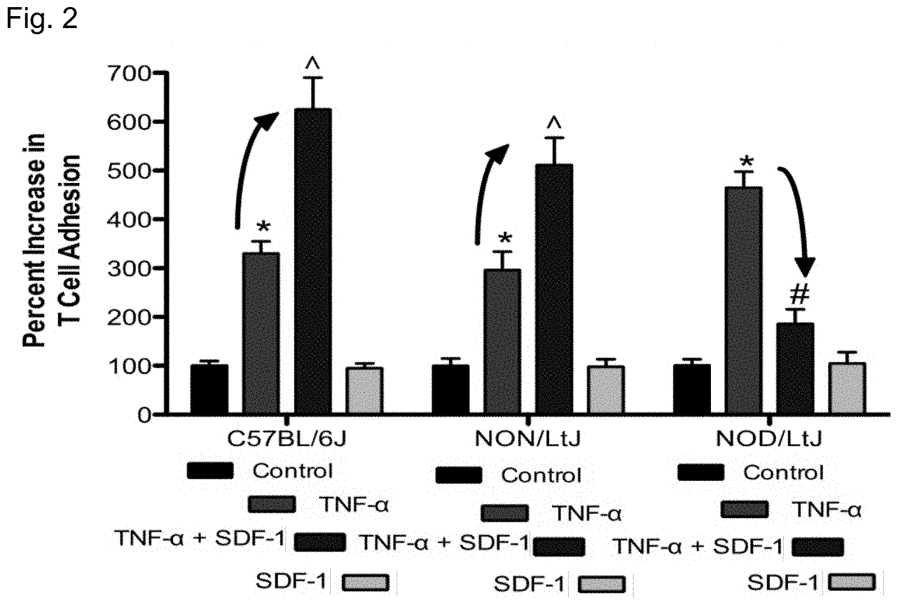

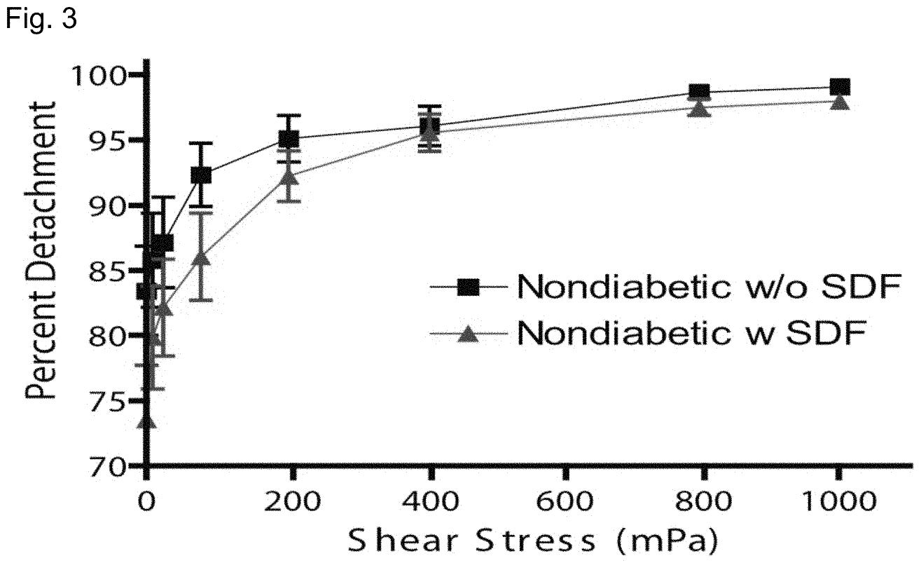

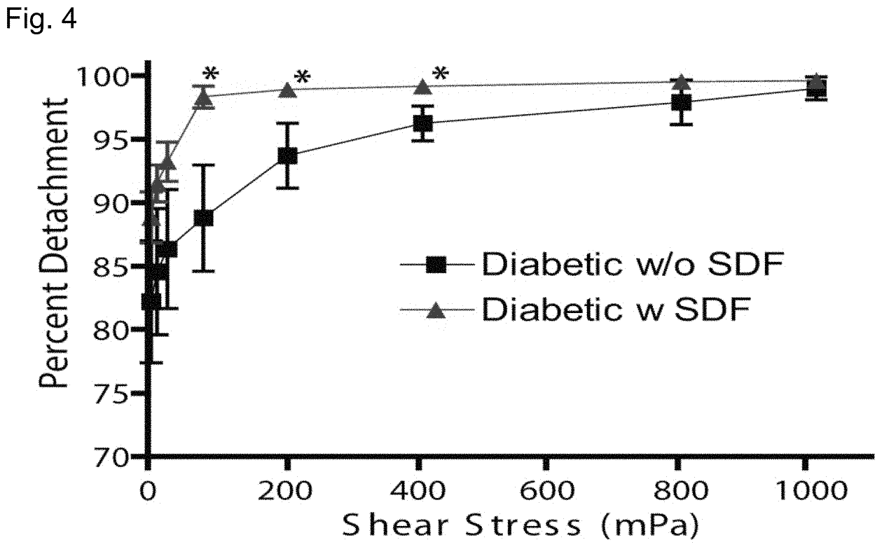

SDF-1α stimulates diabetogenic T cell chemorepulsion: a novel mechanism for potential therapeutic intervention in T1D. Chemokines may be differentially expressed in T1D, which play a role in leukocyte infiltration and adhesion. SDF-1α plays a key role in leukocyte adhesion by activating leukocyte integrins to higher affinity state that promotes their clustering. Due to this effect, SDF-1α has been viewed as a universally potent chemoattractant for leukocyte recruitment at low nanomolar concentrations. However, the inventors discovered novel mechanisms of autoimmune T cell chemorepulsion responses in autoimmune NOD/LtJ diabetic mice and in human T1D T cells at SDF-1α concentrations ranging from 1-100 nM. Data in FIG. 2 illustrate that SDF-1α (50 ng/ml) selectively prevents NOD CD3 T cell adhesion to TNF-α activated islet endothelial cells compared to NON or C57 T cells. Likewise, FIGS. 3 and 4 show that SDF-1α pretreatment selectively stimulated human T1D T cell detachment from fresh human pancreas tissue under physiological shear stress, but not T cell detachment of non-T1D subjects. These data reveal diabetogenic T cell recruitment responses via SDF-1α that represent one leg of a unique therapeutic approach for T1D. Consistent with this notion, it has been shown that SDF-1α/CXCR4 signaling protects against autoimmune diabetes in NOD mice as inhibition of CXCR4 activity by AMD3100 exacerbated adoptive transfer of diabetes.

SDF-1α stimulated diabetogenic T cell chemorepulsion is in response CXCR4 and Robo-1 co-stimulation. The inventors discovered chemorepulsion was in response to co-stimulation of CXCR4 and Robo-1 through cooperative binding of their respective ligands SDF-1α and Slit-2. Interestingly, increased T cell CXCR4 and Robo-1 expression was discovered in both NOD/LtJ mice and patients with T1D, suggesting a unique molecular target for receptor co-stimulation agonists. Robo-1/Slit-2 plays a role in neuronal repellant and axonal guidance in the central nervous system. Slit-2/Robo-1 also has selective chemorepulsion of autoimmune diabetic T cell adhesion. Advantageously, the human and mouse SLIT-2 LRR domain peptides sequences are highly similar. Protein BLAST analysis of the entire Slit2 sequence showed a 96.2% homology between human and mouse slit2 isoform 1, 96.5% homology for isoform 2, and 96.7% homology for isoform 3. Moreover, the LRR domain 2 (D2) peptides and the SDF-1α agonist peptide to be used in constructing the bi-functional peptide are 100% homologous between human and mouse, thereby allowing maximal potential of cross-translation to human T1D patients.

Bifunctional peptide molecules have been used to inhibit antigen presentation through disruption of conjugates between antigenic and cell adhesion peptides. The disclosed SaS-biF-PCs is unique in that this bifunctional peptide will modulate the immune response by selectively inhibiting autoimmune T cell adhesion in T1D and significantly deviates from that of other broad-spectrum immunosuppressive agents used for multiple autoimmune conditions (e.g., rheumatoid arthritis, multiple sclerosis, lupus, e.t.c.).

Diabetic autoreactive T cell recruitment is differentially regulated from normal T cell recruitment. Such findings provide several new possibilities for modulation of autoreactive immune cell recruitment that could provide a selective means with which to alter the development of autoimmune diabetes. The inventors believe that taking advantage of the identified chemorepulsion response using bifunctional peptide conjugates—SaS-biF-PCs offers novel therapeutics to a historically long unmet need of T1D treatment.

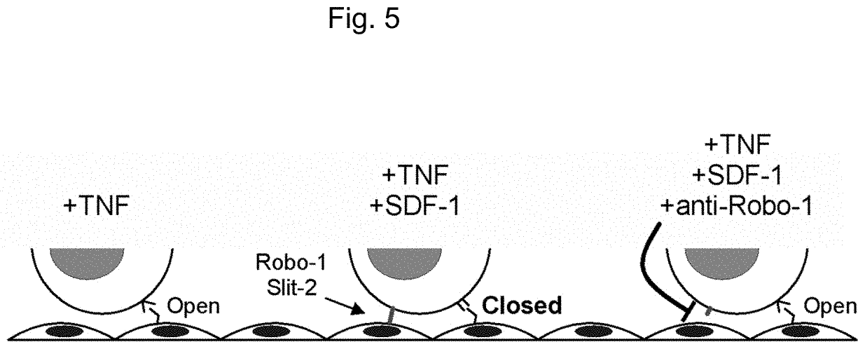

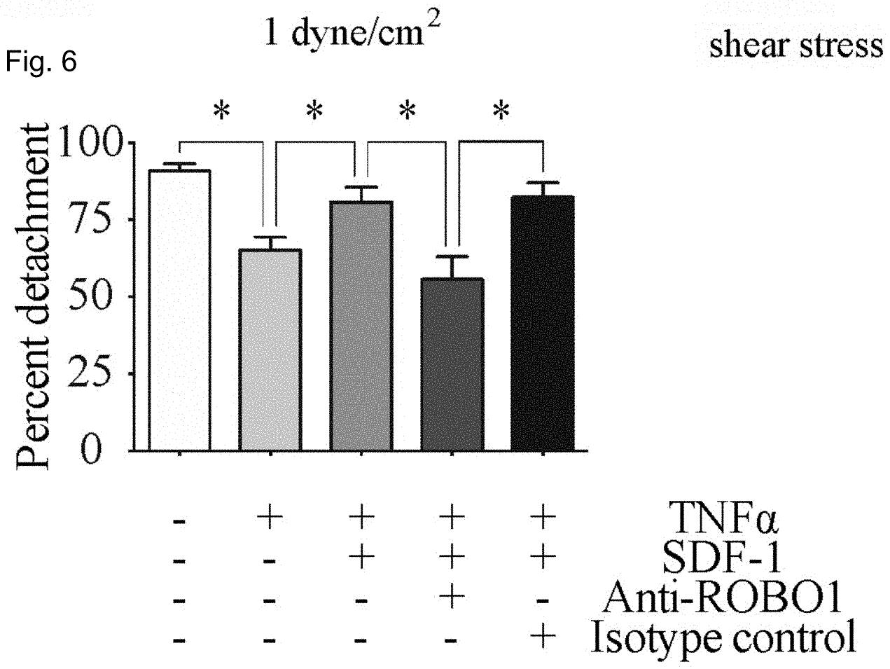

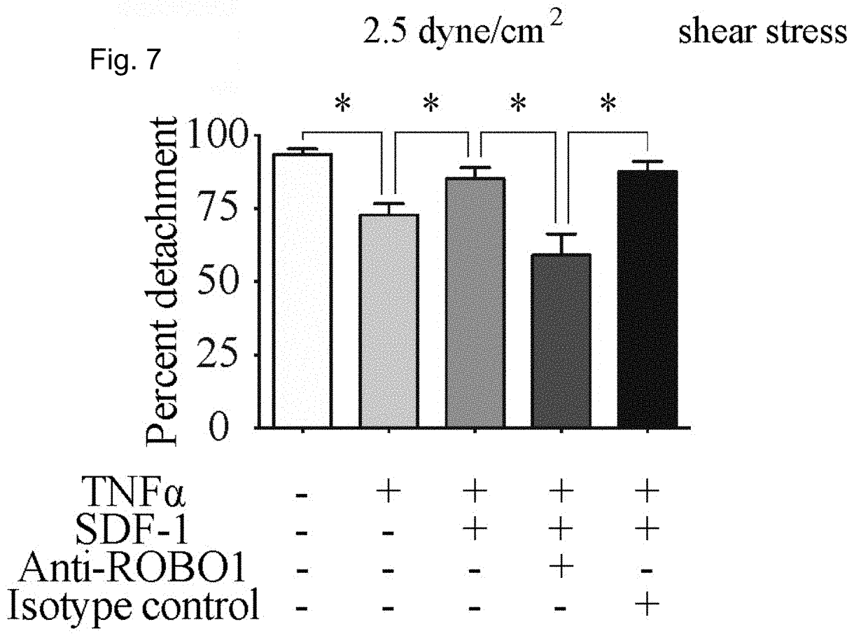

T cell Robo-1 expression governs SDF-1 mediated detachment: The inventors examined whether Robo-1 immuno-neutralization could affect SDF-1α mediated chemorepulsion of NOD/LtJ CD3 T cells. FIGS. 5-7 show results from hydrodynamic shear detachment assays of T cells off of TNF-α activated islet endothelial cells. FIG. 5 illustration indicates molecular events. FIGS. 6 and 7 illustrate the effect of Robo-1 antibody on T cell detachment at low and higher shear rates (1 and 2.5 dyne/cm², respectively). Antibody blockade of Robo-1 binding completely reversed SDF-1α mediated NOD/LtJ T cell enhanced sensitivity to chemorepulsion, implicating T cell Robo-1 expression as critical for the chemorepulsive response.

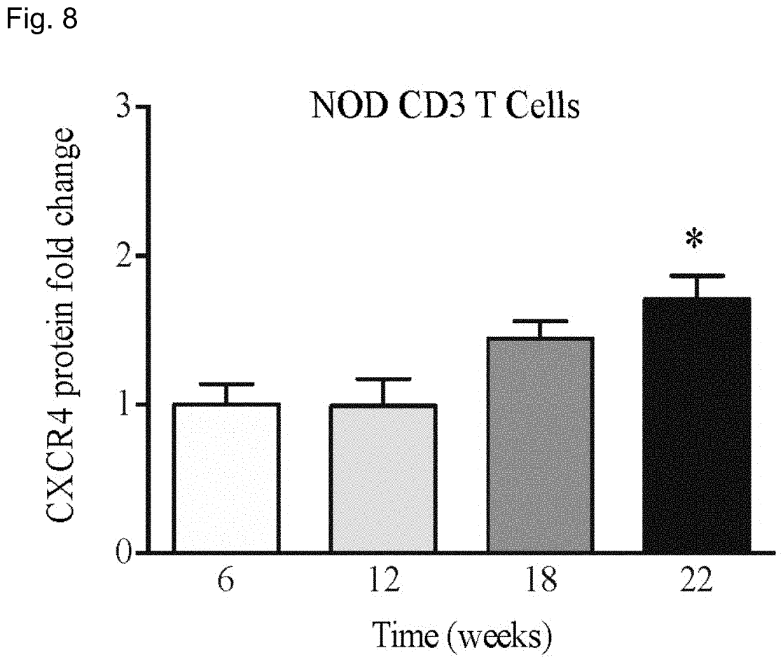

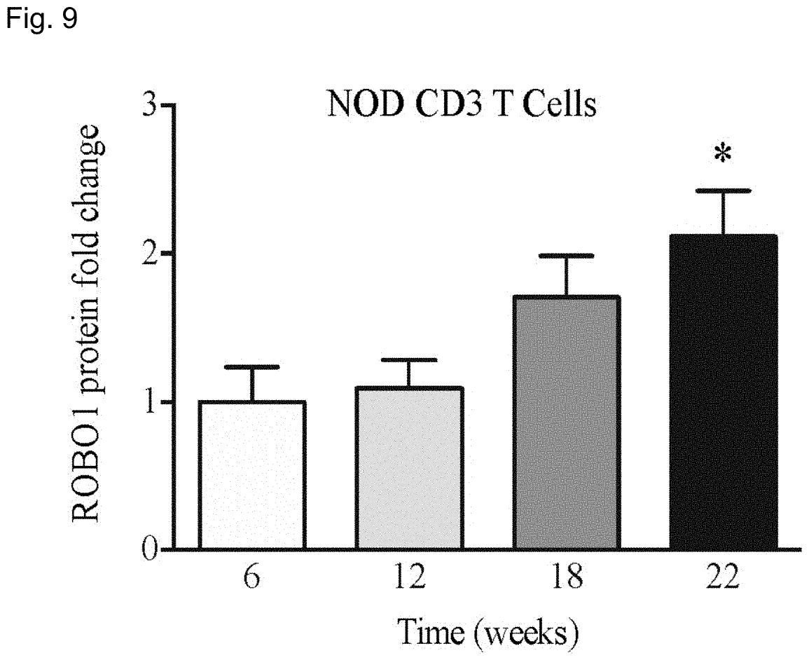

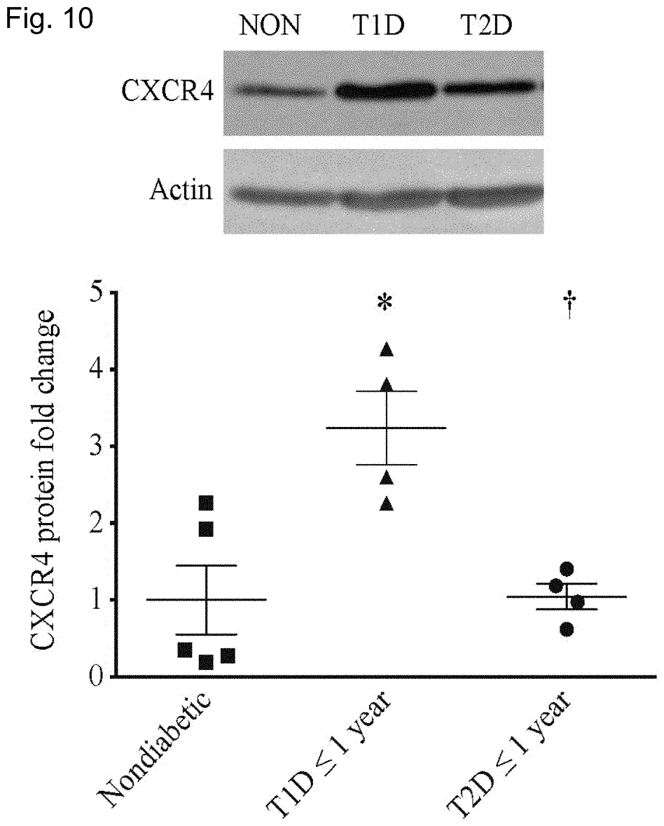

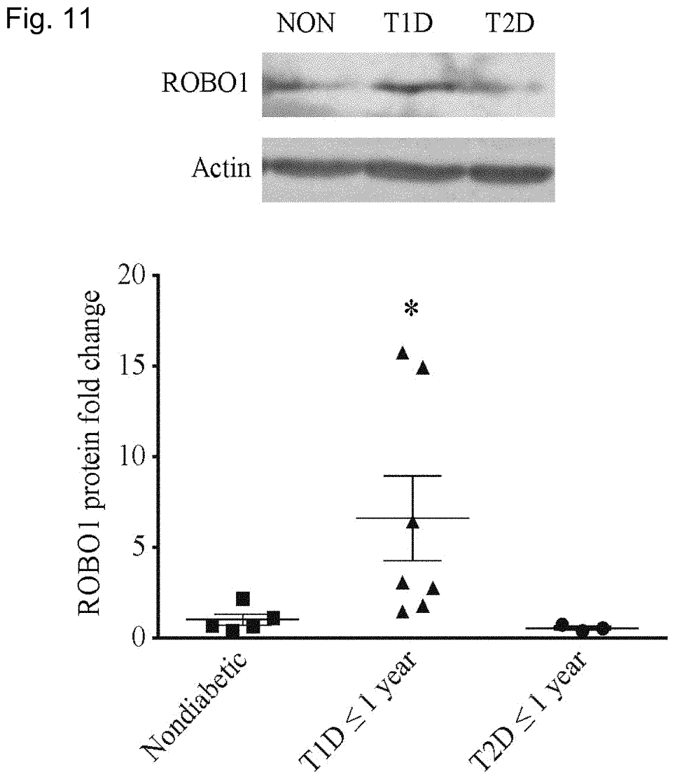

Robo-1 and CXCR4 expression is increased in human diabetic T cells: The inventors next examined whether changes in T cell Robo-1 and CXCR4 expression may be increased in T cells from NOD mice and patients with T1D. FIGS. 8 and 9 illustrate increased T cell protein expression of Robo-1 and CXCR4 from NOD mice over time. Similarly, T cells from T1D subjects diagnosed less than 1 year showed significantly elevated expression of both Robo-1 and CXCR4. FIGS. 10 and 11 demonstrate that the increase in CXCR4 and Robo-1 expression is unique to the autoimmune process and not likely influenced by metabolic dysfunction as increased expression of these molecules were not observed in age matched patients with T2D. These data indicate that similar chemorepulsion pathways are intact in human T1D T cells as seen in NOD mice.

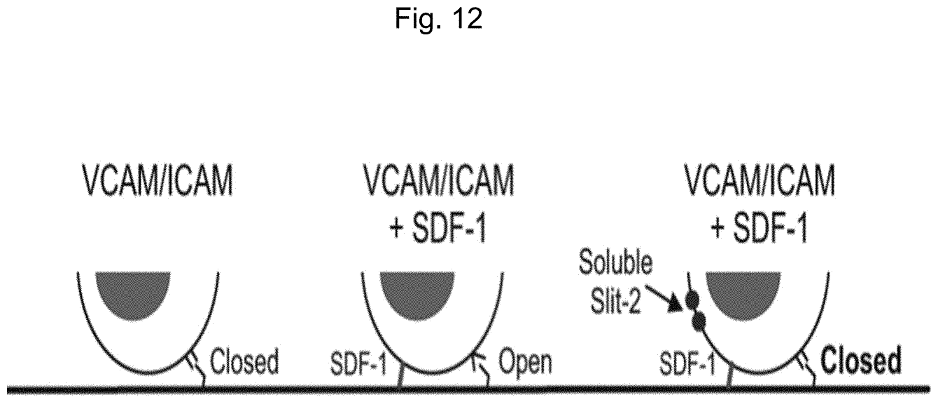

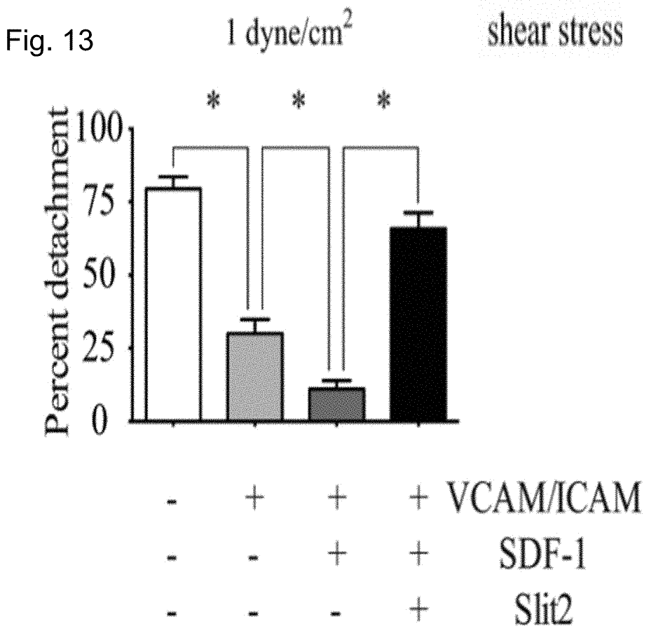

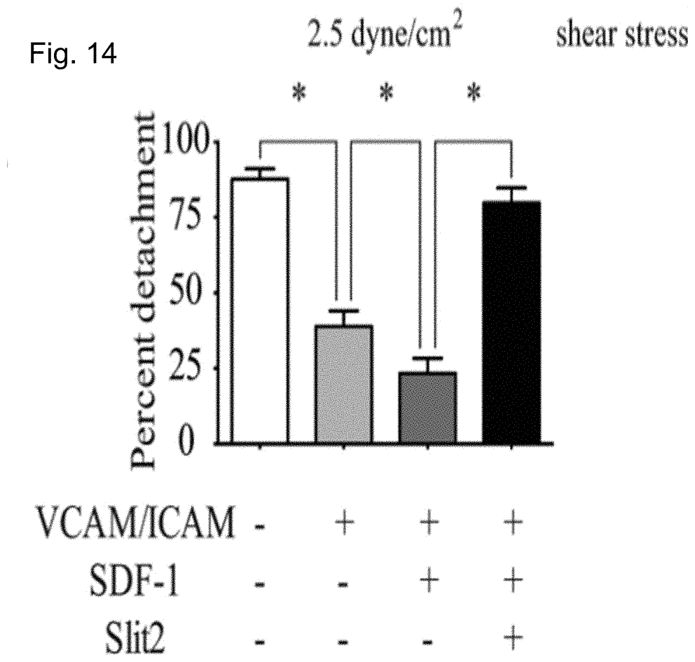

Slit-2 peptide stimulates SDF-1 mediated NOD/LtJ T cell detachment: The inventors next examined whether Slit2 engagement of NOD/LtJ T cell Robo-1 could facilitate SDF-1 mediated chemorepulsion. To study this in an unambiguous way, recombinant Fc chimera endothelial cell adhesion molecules ICAM-1 and VCAM-1 was used to coat the surface of parallel plate flow chambers with or without SDF-1α to precisely control the density and presentation of adhesion molecules and chemokine for adhesive interactions. FIGS. 12-14 show that NOD/LtJ CD3 T cell shear mediated detachment is significantly reduced when bound to plates with ICAM-1 and VCAM-1 and is even more resistant to shear detachment in the presence of SDF-1 (FIG. 13). However, addition of a Slit2 peptide stimulated near complete detachment of NOD T cells under lower and higher shear stress (FIGS. 13-14). Slit2 by itself was unable to stimulate detachment from ICAM-1 and VCAM-1 alone, highlighting the cooperative importance between SDF-1α and Robo-1/Slit2. The illustration of FIG. 12 shows putative integrin changes involved during chemorepulsion.

The inventors believe that the evidence suggests selective dual engagement of CXCR4 and Robo-1 on diabetogenic T cells by a bi-functional peptide conjugate agonist containing CXCL12 and Slit-2 ligand binding domains will stimulate chemorepulsion of diabetogenic T-cell firm adhesion to cell adhesion molecules under hydrodynamic flow conditions. Importantly, as discussed above, homology between mouse and human SLIT-2 and SDF-1α domains to be used are 100%.

Design and synthesis of Slit-2 and SDF-1α bifunctional peptide conjugates (SaS-Bi-PCs). The rationale behind designing SaS-Bi-PCs is that the bifunctional peptide conjugate molecules will bind simultaneously to their respective receptors to modulate the necessary signals to alter the T cell adhesion. Though a sample of SaS-BiF-PCs are disclosed, further modeling and refinement of the bi-functional peptide conjugates for efficiently engaging respective ligand receptors CXCR4 & Robo-1 are anticipated and considered within the scope of the disclosed invention. After modeling, bi-functional peptide conjugate (BPC) molecules will be synthesized using the known agonist peptide region of SDF-1α (e.g., AYWKENKEQ) that is linked to different Slit-2 peptide binding sites for Robo-1. It has been shown that Slit-2 peptides (LRR-7, -8, -9, -10, -11) containing LRR domain (Table 1) can inhibit attachment of NOD/LtJ T cells from recombinant endothelial cell adhesion molecules in conjunction with SDF-1α.

TABLE 1

| Peptide Name | Peptide Sequence |
|---|---|
| LRR-7 | TITEIRLEQN |
| LRR-8 | LRRIDLSNN |
| LRR-9 | LNSLVLYGN |
| LRR-10 | LQLLLLNAN |
| LRR-11 | LNLLSLYDN |
| SDF-1α | AYWKENKEQ |
| SaS-BiF-PC-1 | Ac-TITEIRLEQN-(Acp-G-Acp-G-Acp)₂- |
| | AYWKENKEQ-NH₂ |
| SaS-BiF-PC-2 | Ac-LRRIDLSNN-(Acp-G-Acp-G-Acp)₂- |
| | AYWKENKEQ-NH₂ |
| SaS-BiF-PC-3 | Ac-LNSLVLYGN-(Acp-G-Acp-G-Acp)₂- |
| | AYWKENKEQ-NH₂ |
| SaS-BiF-PC-4 | Ac-LQLLLLNAN-(Acp-G-Acp-G-Acp)₂- |
| | AYWKENKEQ-NH₂ |
| SaS-BiF-PC-5 | Ac-LNLLSLYDN-(Acp-G-Acp-G-Acp)₂- |
| | AYWKENKEQ-NH₂ |
| SaS-BiF-PC-6 | AC-LQLLLLNAN-(PEG3-PEG3)₂- |
| | AYWKENKEQ-NH₂ |

Acp = Amino caproate; G = Glycine; PEG3 = Polyethylene Glycols.

It is anticipated that five peptides from Slit-2 known to interact with Robo-1 will be synthesized and conjugated with the SDF-1α peptide (termed SaS-BiF-PC-1 to SaS-BiF-PC-6, see Table 1). The two peptides will be separated by a linker, which preferably spans ˜40 A° to allow the two peptides to bind both receptors on the cell surface. An established linker [(Acp-G-Acp-G-Acp)₂] from successful bi-functional peptides molecules will be used in one embodiment to ensure the the efficacy of SaS-BiF-PC molecules. In addition, the peptides may be acetylated and/or amidated at the N- and C- terminus, respectively, or otherwise stabilized to improve their plasma stability. Furthermore, to follow binding of SaS-BiF-PC to both receptors, they may be tagged to reporters. To achieve this, one of the linker Gly residues may be substituted with a Lys residue and the side chain amino group of the Lys residue will be attached with a fluorescence tag such as fluorescein isothiocyanate (FITC) or phycoerythrin (PE). The binding of the SaS-BiF-PC to both receptors can be determined using colocalization studies of the fluorescence-labeled BPC and antibody to both receptors carrying a different fluorescence tag.

Molecular modeling studies may be performed to gain insight to Sas-BiF-PC binding affinities using in silico methodologies. Further, the experiments to study the binding between BPC and their respective receptors may be done using a combination of molecular dynamics simulations and molecular docking experiments.

There is a possibility that the peptide generated may be hydrophobic; in such case, PEG linkers will be preferably be used to improve the solubility of the peptide and lower the immunogenicity of the peptide. In addition, Lysine residues may be incorporated in the middle of the linker to improve the solubility of the SaS-BiF-PC peptides, and lower potential allergic response of the mammal or human to the peptide. Binding interactions may be further validated through proximity ligation assay evaluation of Robo-1 & CXCR4 cell surface location.

As used herein, the term “delayed release” includes a pharmaceutical preparation, e.g., an orally administered formulation, which passes through the stomach substantially intact and dissolves in the small and/or large intestine (e.g., the colon). In some embodiments, delayed release of the active agent (e.g., a therapeutic as described herein) results from the use of an enteric coating of an oral medication (e.g., an oral dosage form).

The term an “effective amount” of an agent, as used herein, is that amount sufficient to effect beneficial or desired results, such as clinical results, and, as such, an “effective amount” depends upon the context in which it is being applied.

The terms “extended release” or “sustained release” interchangeably include a drug formulation that provides for gradual release of a drug over an extended period of time, e.g., 6-12 hours or more, compared to an immediate release formulation of the same drug. Preferably, although not necessarily, results in substantially constant blood levels of a drug over an extended time period that are within therapeutic levels and fall within a peak plasma concentration range that is between, for example, 0.05-10 μM, 0.1-10 μM, 0.1-5.0 μM, or 0.1-1 μM.

As used herein, the terms “formulated for enteric release” and “enteric formulation” include pharmaceutical compositions, e.g., oral dosage forms, for oral administration able to provide protection from dissolution in the high acid (low pH) environment of the stomach. Enteric formulations can be obtained by, for example, incorporating into the pharmaceutical composition a polymer resistant to dissolution in gastric juices. In some embodiments, the polymers have an optimum pH for dissolution in the range of approx. 5.0 to 7.0 (“pH sensitive polymers”). Exemplary polymers include methacrylate acid copolymers that are known by the trade name Eudragit® (e.g., Eudragit® L100, Eudragit® S100, Eudragit® L-30D, Eudragit® FS 30D, and Eudragit® L100-55), cellulose acetate phthalate, cellulose acetate trimellitiate, polyvinyl acetate phthalate (e.g., Coateric®, hydroxyethylcellulose phthalate, hydroxypropyl methylcellulose phthalate, or shellac, or an aqueous dispersion thereof. Aqueous dispersions of these polymers include dispersions of cellulose acetate phthalate (Aquateric®) or shellac (e.g., MarCoat 125 and 125N). An enteric formulation reduces the percentage of the administered dose released into the stomach by at least 50%, 60%, 70%, 80%, 90%, 95%, or even 98% in comparison to an immediate release formulation. Where such a polymer coats a tablet or capsule, this coat is also referred to as an “enteric coating.”

The term “immediate release” includes where the agent (e.g., therapeutic), as formulated in a unit dosage form, has a dissolution release profile under in vitro conditions in which at least 55%, 65%, 75%, 85%, or 95% of the agent is released within the first two hours of administration to, e.g., a human. Desirably, the agent formulated in a unit dosage has a dissolution release profile under in vitro conditions in which at least 50%, 65%, 75%, 85%, 90%, or 95% of the agent is released within the first 30 minutes, 45 minutes, or 60 minutes of administration.

The term “pharmaceutical composition,” as used herein, includes a composition containing a compound described herein (e.g., T1D, or any pharmaceutically acceptable salt, solvate, or prodrug thereof), formulated with a pharmaceutically acceptable excipient, and typically manufactured or sold with the approval of a governmental regulatory agency as part of a therapeutic regimen for the treatment of disease in a mammal.

Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, includes any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, hydroxypropyl cellulose, hydroxypropyl methylcellulose, lactose, magnesium stearate, maltitol, maltose, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable prodrugs” as used herein, includes those prodrugs of the compounds of the present invention which are, within the scope of sound medical judgment, suitable for use in contact with the tissues of humans and animals with undue toxicity, irritation, allergic response, and the like, commensurate with a reasonable benefit/risk ratio, and effective for their intended use, as well as the zwitterionic forms, where possible, of the compounds of the invention.

The term “pharmaceutically acceptable salt,” as use herein, includes those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds of the invention or separately by reacting the free base group with a suitable organic or inorganic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The terms “pharmaceutically acceptable solvate” or “solvate,” as used herein, includes a compound of the invention wherein molecules of a suitable solvent are incorporated in the crystal lattice. A suitable solvent is physiologically tolerable at the administered dose. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

The term “prevent,” as used herein, includes prophylactic treatment or treatment that prevents one or more symptoms or conditions of a disease, disorder, or conditions described herein (e.g., a T1D). Treatment can be initiated, for example, prior to (“pre-exposure prophylaxis”) or following (“post-exposure prophylaxis”) an event that precedes the onset of the disease, disorder, or conditions. Treatment that includes administration of a compound of the invention, or a pharmaceutical composition thereof, can be acute, short-term, or chronic. The doses administered may be varied during the course of preventive treatment.

The term “prodrug,” as used herein, includes compounds which are rapidly transformed in vivo to the parent compound of the above formula. Prodrugs also encompass bioequivalent compounds that, when administered to a human, lead to the in vivo formation of therapeutic. A thorough discussion is provided in T. Higuchi and V. Stella, Pro-drugs as Novel Delivery Systems, Vol. 14 of the A.C.S. Symposium Series, and Edward B. Roche, ed., Bioreversible Carriers in Drug Design, American Pharmaceutical Association and Pergamon Press, 1987, each of which is incorporated herein by reference. Preferably, prodrugs of the compounds of the present invention are pharmaceutically acceptable.

As used herein, and as well understood in the art, “treatment” includes an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e. not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Treatment” can also mean prolonging survival as compared to expected survival if not receiving treatment. As used herein, the terms “treating” and “treatment” can also include delaying the onset of, impeding or reversing the progress of, or alleviating either the disease or condition to which the term applies, or one or more symptoms of such disease or condition.

The term “unit dosage forms” includes physically discrete units suitable as unitary dosages for human subjects and other mammals, each unit containing a predetermined quantity of active material calculated to produce the desired therapeutic effect, in association with any suitable pharmaceutical excipient or excipients.

As used herein, the term “plasma concentration” includes the amount of therapeutic present in the plasma of a treated subject (e.g., as measured in a rabbit using an assay described below or in a human).

### Pharmaceutical Compositions

The methods described herein can also include the administrations of pharmaceutically acceptable compositions that include the therapeutic, or a pharmaceutically acceptable salt, solvate, or prodrug thereof. When employed as pharmaceuticals, any of the present compounds can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

This invention also includes pharmaceutical compositions which can contain one or more pharmaceutically acceptable carriers. In making the pharmaceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, such as preservatives.

The therapeutic agents of the invention can be administered alone, or in a mixture, in the presence of a pharmaceutically acceptable excipient or carrier. The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 22^{nd} Ed., Gennaro, Ed., Lippencott Williams & Wilkins (2012), a well-known reference text in this field, and in the USP/NF (United States Pharmacopeia and the National Formulary), each of which is incorporated by reference. In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. Other exemplary excipients are described in Handbook of Pharmaceutical Excipients, 8^{th} Edition, Sheskey et al., Eds., Pharmaceutical Press (2017), which is incorporated by reference.

The methods described herein can include the administration of a therapeutic, or prodrugs or pharmaceutical compositions thereof, or other therapeutic agents. Exemplary therapeutics include those that bind both Robo-1 and CXCR-4 (including a Slit-2 and SDF-1α bifuncitional peptide conjugate).

The pharmaceutical compositions can be formulated so as to provide immediate, extended, or delayed release of the active ingredient after administration to the patient by employing procedures known in the art.

The compositions can be formulated in a unit dosage form, each dosage containing, e.g., 0.1-500 mg of the active ingredient. For example, the dosages can contain from about 0.1 mg to about 50 mg, from about 0.1 mg to about 40 mg, from about 0.1 mg to about 20 mg, from about 0.1 mg to about 10 mg, from about 0.2 mg to about 20 mg, from about 0.3 mg to about 15 mg, from about 0.4 mg to about 10 mg, from about 0.5 mg to about 1 mg; from about 0.5 mg to about 100 mg, from about 0.5 mg to about 50 mg, from about 0.5 mg to about 30 mg, from about 0.5 mg to about 20 mg, from about 0.5 mg to about 10 mg, from about 0.5 mg to about 5 mg; from about 1 mg from to about 50 mg, from about 1 mg to about 30 mg, from about 1 mg to about 20 mg, from about 1 mg to about 10 mg, from about 1 mg to about 5 mg; from about 5 mg to about 50 mg, from about 5 mg to about 20 mg, from about 5 mg to about 10 mg; from about 10 mg to about 100 mg, from about 20 mg to about 200 mg, from about 30 mg to about 150 mg, from about 40 mg to about 100 mg, from about 50 mg to about 100 mg of the active ingredient, from about 50 mg to about 300 mg, from about 50 mg to about 250 mg, from about 100 mg to about 300 mg, or , from about 100 mg to about 250 mg of the active ingredient. For preparing solid compositions such as tablets, the principal active ingredient is mixed with one or more pharmaceutical excipients to form a solid bulk formulation composition containing a homogeneous mixture of a compound of the present invention. When referring to these bulk formulation compositions as homogeneous, the active ingredient is typically dispersed evenly throughout the composition so that the composition can be readily subdivided into equally effective unit dosage forms such as tablets and capsules. This solid bulk formulation is then subdivided into unit dosage forms of the type described above containing from, for example, 0.1 to about 500 mg of the active ingredient of the present invention.

### Compositions for Oral Administration

The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Formulations for oral administration may also be presented as chewable tablets, as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration vs time profile. In one example, controlled release is obtained by appropriate selection of various formulation parameters and ingredients, including, e.g., various types of controlled release compositions and coatings. Thus, the drug is formulated with appropriate excipients into a pharmaceutical composition that, upon administration, releases the drug in a controlled manner. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palm itostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions suitable for oral mucosal administration (e.g., buccal or sublingual administration) include tablets, lozenges, and pastilles, where the active ingredient is formulated with a carrier, such as sugar, acacia, tragacanth, or gelatin and glycerine.

### Coatings

The pharmaceutical compositions formulated for oral delivery, such as tablets or capsules of the present invention can be coated or otherwise compounded to provide a dosage form affording the advantage of delayed or extended release. The coating may be adapted to release the active drug substance in a predetermined pattern (e.g., in order to achieve a controlled release formulation) or it may be adapted not to release the active drug substance until after passage of the stomach, e.g., by use of an enteric coating (e.g., polymers that are pH-sensitive (“pH controlled release”), polymers with a slow or pH-dependent rate of swelling, dissolution or erosion (“time-controlled release”), polymers that are degraded by enzymes (“enzyme-controlled release” or “biodegradable release”) and polymers that form firm layers that are destroyed by an increase in pressure (“pressure-controlled release”)). Exemplary enteric coatings that can be used in the pharmaceutical compositions described herein include sugar coatings, film coatings (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or coatings based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose. Furthermore, a time delay material such as, for example, glyceryl monostearate or glyceryl distearate, may be employed.

For example, the tablet or capsule can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release.

When an enteric coating is used, desirably, a substantial amount of the drug is released in the lower gastrointestinal tract.

In addition to coatings that effect delayed or extended release, the solid tablet compositions may include a coating adapted to protect the composition from unwanted chemical changes (e.g., chemical degradation prior to the release of the active drug substance). The coating may be applied on the solid dosage form in a similar manner as that described in Encyclopedia of Pharmaceutical Technology, vols. 5 and 6, Eds. Swarbrick and Boyland, 2000.

### Parenteral Administration

Within the scope of the present invention are also parenteral depot systems from biodegradable polymers. These systems are injected or implanted into the muscle or subcutaneous tissue and release the incorporated drug over extended periods of time, ranging from several days to several months. Both the characteristics of the polymer and the structure of the device can control the release kinetics which can be either continuous or pulsatile. Polymer-based parenteral depot systems can be classified as implants or microparticles. The former are cylindrical devices injected into the subcutaneous tissue whereas the latter are defined as spherical particles in the range of 10-100 μm. Extrusion, compression or injection molding are used to manufacture implants whereas for microparticles, the phase separation method, the spray-drying technique and the water-in-oil-in-water emulsion techniques are frequently employed. The most commonly used biodegradable polymers to form microparticles are polyesters from lactic and/or glycolic acid, e.g. poly(glycolic acid) and poly(L-lactic acid) (PLG/PLA microspheres). Of particular interest are in situ forming depot systems, such as thermoplastic pastes and gelling systems formed by solidification, by cooling, or due to the sol-gel transition, cross-linking systems and organogels formed by amphiphilic lipids. Examples of thermosensitive polymers used in the aforementioned systems include, N-isopropylacrylamide, poloxamers (ethylene oxide and propylene oxide block copolymers, such as poloxamer 188 and 407), poly(N-vinyl caprolactam), poly(siloethylene glycol), polyphosphazenes derivatives and PLGA-PEG-PLGA.

### Mucosal Drug Delivery

Mucosal drug delivery (e.g., drug delivery via the mucosal linings of the nasal, rectal, vaginal, ocular, or oral cavities) can also be used in the methods described herein. Methods for oral mucosal drug delivery include sublingual administration (via mucosal membranes lining the floor of the mouth), buccal administration (via mucosal membranes lining the cheeks), and local delivery (Harris et al., Journal of Pharmaceutical Sciences, 81(1): 1-10, 1992).

Oral transmucosal absorption is generally rapid because of the rich vascular supply to the mucosa and allows for a rapid rise in blood concentrations of the therapeutic.

For buccal administration, the compositions may take the form of, e.g., tablets, lozenges, etc. formulated in a conventional manner. Permeation enhancers can also be used in buccal drug delivery. Exemplary enhancers include 23-lauryl ether, aprotinin, azone, benzalkonium chloride, cetylpyridinium chloride, cetyltrimethylammonium bromide, cyclodextrin, dextran sulfate, lauric acid, lysophosphatidylcholine, methol, methoxysalicylate, methyloleate, oleic acid, phosphatidylcholine, polyoxyethylene, polysorbate 80, sodium EDTA, sodium glycholate, sodium glycodeoxycholate, sodium lauryl sulfate, sodium salicylate, sodium taurocholate, sodium taurodeoxycholate, sulfoxides, and alkyl glycosides. Bioadhesive polymers have extensively been employed in buccal drug delivery systems and include cyanoacrylate, polyacrylic acid, hydroxypropyl methylcellulose, and poly methacrylate polymers, as well as hyaluronic acid and chitosan.

Liquid drug formulations (e.g., suitable for use with nebulizers and liquid spray devices and electrohydrodynamic (EHD) aerosol devices) can also be used. Other methods of formulating liquid drug solutions or suspension suitable for use in aerosol devices are known to those of skill in the art (see, e.g., Biesalski, U.S. Pat. No. 5,112,598, and Biesalski, U.S. Pat. No. 5,556,611).

Formulations for sublingual administration can also be used, including powders and aerosol formulations. Exemplary formulations include rapidly disintegrating tablets and liquid-filled soft gelatin capsules.

### Dosing Regimes

The present methods for treating T1D are carried out by administering a therapeutic for a time and in an amount sufficient to result in diabetogenic T-cell chemorepulsion.

The amount and frequency of administration of the compositions can vary depending on, for example, what is being administered, the state of the patient, and the manner of administration. In therapeutic applications, compositions can be administered to a patient suffering from T1D in an amount sufficient to relieve or least partially relieve the symptoms of the T1D and its complications. The dosage is likely to depend on such variables as the type and extent of progression of the T1D, the severity of the T1D, the age, weight and general condition of the particular patient, the relative biological efficacy of the composition selected, formulation of the excipient, the route of administration, and the judgment of the attending clinician. Effective doses can be extrapolated from dose-response curves derived from in vitro or animal model test system. An effective dose is a dose that produces a desirable clinical outcome by, for example, improving a sign or symptom of the T1D or slowing its progression.

The amount of therapeutic per dose can vary. For example, a subject can receive from about 0.1 μg/kg to about 10,000 μg/kg. Generally, the therapeutic is administered in an amount such that the peak plasma concentration ranges from 150 nM-250 μM.

Exemplary dosage amounts can fall between 0.1-5000 μg/kg, 100-1500 μg/kg, 100-350 μg/kg, 340-750 μg/kg, or 750-1000 μg/kg. Exemplary dosages can 0.25, 0.5, 0.75, 1°, or 2 mg/kg. In another embodiment, the administered dosage can range from 0.05-5 mmol of therapeutic (e.g., 0.089-3.9 mmol) or 0.1-50 μmol of therapeutic (e.g., 0.1-25 μmol or 0.4-20 μmol).

The plasma concentration of therapeutic can also be measured according to methods known in the art. Exemplary peak plasma concentrations of therapeutic can range from 0.05-10 μM, 0.1-10 μM, 0.1-5.0 μM, or 0.1-1 μM. Alternatively, the average plasma levels of therapeutic can range from 400-1200 μM (e.g., between 500-1000 μM) or between 50-250 μM (e.g., between 40-200 μM). In some embodiments where sustained release of the drug is desirable, the peak plasma concentrations (e.g., of therapeutic) may be maintained for 6-14 hours, e.g., for 6-12 or 6-10 hours. In other embodiments where immediate release of the drug is desirable, the peak plasma concentration (e.g., of therapeutic) may be maintained for, e.g., 30 minutes.

The frequency of treatment may also vary. The subject can be treated one or more times per day with therapeutic (e.g., once, twice, three, four or more times) or every so-many hours (e.g., about every 2, 4, 6, 8, 12, or 24 hours). Preferably, the pharmaceutical composition is administered 1 or 2 times per 24 hours. The time course of treatment may be of varying duration, e.g., for two, three, four, five, six, seven, eight, nine, ten or more days. For example, the treatment can be twice a day for three days, twice a day for seven days, twice a day for ten days. Treatment cycles can be repeated at intervals, for example weekly, bimonthly or monthly, which are separated by periods in which no treatment is given. The treatment can be a single treatment or can last as long as the life span of the subject (e.g., many years).

### Kits

Any of the pharmaceutical compositions of the invention described herein can be used together with a set of instructions, i.e., to form a kit. The kit may include instructions for use of the pharmaceutical compositions as a therapy as described herein. For example, the instructions may provide dosing and therapeutic regimes for use of the compounds of the invention to reduce symptoms and/or underlying cause of the T1D.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items while only the terms “consisting of” and “consisting only of” are to be construed in the limitative sense.

Wherefore, I/we claim:

1. A pharmaceutical composition comprising:
a Slit-2 and SDF-1α bifuncitional peptide conjugate having a Slit-2 ligand binding domain and an SDF-1α ligand binding domain.

2. The pharmaceutical composition of claim 1 further comprising a linker that spans about 40 A°.

3. The pharmaceutical composition of claim 1 further comprising a linker is one of (Acp-G-Acp-G-Acp)₂ and (PEG3-PEG3)₂, where “Acp” is Amino caproate, “G” is Glycine, and “PEG” is Polyethylene Glycol.

4. The pharmaceutical composition of claim 1 wherein the SDF-1α ligand binding domain includes AYWKENKEQ.

5. The pharmaceutical composition of claim 1 wherein the Slit-2 ligand binding domain containing an LLR domain.

6. The pharmaceutical composition of claim 1 wherein the Slit-2 ligand binding domain containing one of TITEIRLEQN, LRRIDLSNN, LNSLVLYGN, LQLLLLNAN, and LNLLSLYDN.

7. The pharmaceutical composition of claim 1 wherein a peptide sequence of the Slit-2 and SDF1α bifuncitional peptide conjugate includes one of Ac-TITEIRLEQN-(Acp-G-Acp-G-Acp)₂-AYWKENKEQ-NH₂, Ac-LRRIDLSNN-(Acp-G-Acp-G-Acp)₂-AYWKENKEQ-NH₂, Ac-LNSLVLYGN-(Acp-G-Acp-G-Acp)₂-AYWKENKEQ-NH₂, Ac-LQLLLLNAN-(Acp-G-Acp-G-Acp)₂-AYWKENKEQ-NH₂, Ac-LNLLSLYDN-(Acp-G-Acp-G-Acp)₂-AYWKENKEQ-NH₂, and AC-LQLLLLNAN-(PEG3-PEG3)₂-AYWKENKEQ-NH₂, where “Acp” is Amino caproate, “G” is Glycine, and “PEG” is Polyethylene Glycol.

8. The pharmaceutical composition of claim 1 wherein the peptide conjugate is one acetylated at an N- terminus, amidated at a C- terminus, and both acetylated at the N- terminus and amidated at the C- terminus.

9. The pharmaceutical composition of claim 1 wherein the peptide conjugate contains a peptide mimetic of SLIT2.

10. The pharmaceutical composition of claim 1 wherein the peptide conjugate contains a peptide mimetic of SDF-1α.

11. A method of treating Type 1 diabetes comprising:
administering to a human mammalian patient a thereapeutic amount of pharmaceutical composition including a Slit-2 and SDF-1α bifuncitional peptide conjugate.

12. The method of claim 11 further comprising the step of administering insulin to the patient.

13. The method of claim 11 further comprising the step of transplanting one of beta cells or a pancreas to the patient.


SEQUENCE LISTING (12 sequences)

SEQ ID NO: 1 - protein, 10 aa, Homo sapiens
TITEIRLEQN

SEQ ID NO: 2 - protein, 9 aa, Homo sapiens
LRRIDLSNN

SEQ ID NO: 3 - protein, 9 aa, Homo sapiens
LNSLVLYGN

SEQ ID NO: 4 - protein, 9 aa, Homo sapiens
LQLLLLNAN

SEQ ID NO: 5 - protein, 9 aa, Homo sapiens
LNLLSLYDN

SEQ ID NO: 6 - protein, 9 aa, Homo sapiens
AYWKENKEQ

SEQ ID NO: 7 - protein, 45 aa, Homo sapiens
ACTITEIRLEQNACPGACPGACPACPGACPGACPAYWKENKEQNH

SEQ ID NO: 8 - protein, 44 aa, Homo sapiens
ACLRRIDLSNNACPGACPGACPACPGACPGACPAYWKENKEQNH

SEQ ID NO: 9 - protein, 44 aa, Homo sapiens
ACLNSLVLYGNACPGACPGACPACPGACPGACPAYWKENKEQNH

SEQ ID NO: 10 - protein, 44 aa, Homo sapiens
ACLQLLLLNANACPGACPGACPACPGACPGACPAYWKENKEQNH

SEQ ID NO: 11 - protein, 44 aa, Homo sapiens
ACLNLLSLYDNACPGACPGACPACPGACPGACPAYWKENKEQNH

SEQ ID NO: 12 - protein, 34 aa, Homo sapiens
ACLQLLLLNANPEGPEGPEGPEGAYWKENKEQNH